(19) United States
(12) Patent Application Publication
(10) Pub. No.: US 2023/0108408 A1
(43) Pub. Date: Apr. 6, 2023

(54) OXALAMIDE SUBSTITUTED HETEROCYCLIC COMPOUNDS AS MODULATORS OF THE ARYL HYDROCARBON RECEPTOR (AHR)

(71) Applicant: PHENEX PHARMACEUTICALS AG, Heidelberg (DE)

(72) Inventors: Olaf Kinzel, Heidelberg (DE); Christoph Steeneck, Heidelberg (DE); Marta Czekanska, Saarbrücken (DE); Sheena Pinto, Heidelberg (DE); Ibrahim Murathan Sektioglu, Heidelberg (DE); Ulrich Deuschle, Speyer (DE)

(73) Assignee: PHENEX PHARMACEUTICALS AG, Heidelberg (DE)

(21) Appl. No.: 17/759,266

(22) PCT Filed: Jan. 22, 2021
(86) PCT No.: PCT/EP2021/051492

§ 371 Date: Jul. 21, 2022

(30) Foreign Application Priority Data
Jan. 23, 2020 (EP) 20153381.7

(51) Int. Cl. C07D 471/04; A61K 45/06

(52) U.S. Cl. CPC C07D 471/04; A61K 45/06

(57) ABSTRACT

The present invention relates to oxalamide substituted heterocyclic compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

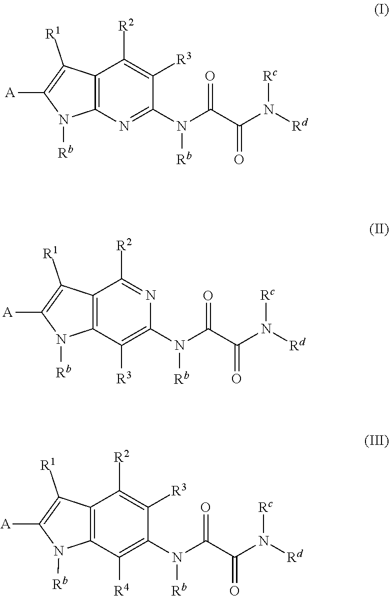

The present invention relates to compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

The aryl hydrocarbon receptor (AhR) is a ligand-modulated transcription factor, belonging to the basic helix-loop-helix PAS (Per-Arnt-Sim homology domain) family, that is expressed in most tissues in mice and humans and known to mediate many of the toxicities of 2,3,7,8-Tetrachlorodibenzo-p-dioxin (TCDD) in mice. The AhR protein is localized in the cytoplasm of eukaryotic cells in complexes with HSP90 and other proteins. Binding of agonistic ligands, such as TCDD, leads to dissociation of AhR from the HSP90 containing complex, transport to the nucleus and association with its heterodimeric partner ARNT. This heterodimeric complex can bind to AhR response elements located in promoter regions of genes such as CYP1A1, CYP1 B1, ALDH3A1, NQO1, UGT1A1 etc. and induces the transcription of such genes in case of very potent and efficacious AhR agonists, such as TCDD.

By regulating the expression of genes involved in xenobiotic transformation (e.g. CYP1A1), the AhR plays a significant role in the detoxification of xenobiotic substances in liver and intestine, which are prominent locations of AhR expression. This activity might be underlying some of the described chemoprevention and tumor suppression effects exerted by AhR. On the other hand, CYP1A1 is known to metabolize some pro-cancerogens, such as benzo(a)pyrene into DNA reactive intermediates leading to mutagenesis and tumor formation (Murray et al. Nat Rev Cancer. 2014 December; 14(12):801-14; Safe et al Toxicol Sci. 2013 September; 135(1):1-16).

In mouse cancer models, knock-down of AhR typically resulted in decreased proliferation and/or invasion and migration of cancer cell lines and overexpression of constitutive active AhR results in vivo in enhanced stomach and liver cancers (Safe et al Toxicol Sci. 2013 September; 135(1):1-16).

The AhR is relatively strongly expressed in intestinal epithelial tissues, lung epithelium and skin. In these tissues the AhR expression is particularly high in cells of lymphoid origin such as T-cells, Dendritic Cells, Langerhans Cells, Macrophages, Mast cells etc. One possible function in these compartments is to integrate signals from the commensal microbiomes in the intestine, the lung and the skin, which are known to produce diverse mixtures of indolic AhR modulators that are thought to balance the responses of the immune system towards the microbiome (Bessede et al., Nature. 2014 Jul. 10; 511(7508):184-90, Zelante et al. Immunity. 2013 Aug. 22; 39(2):372-85, Romani et al., Eur J Immunol. 2014 November; 44(11):3192-200).

The expression of AhR has been found to be constitutive active in advanced human prostate cancer (Richmond et al., 2014, PLoS ONE 9(4): e95058), overexpressed in breast cancer (Li et al., Int J Clin Exp Pathol. 2014 Oct. 15; 7(11):7931) and pancreas cancer (Koliopanos et al., Oncogene. 2002 Sep. 5; 21(39):6059-70). Modulation of the AhR pathway activity by small molecule modulators might be beneficial for some of these devastating diseases with very limited treatment options.

In a recently published Patent Application US 2016/01752278 by the Trustees of Boston University, novel small molecule agents characterized as AhR modulators are being claimed for inhibiting cancer cell proliferation, tumor cell invasion and metastasis.

AhR modulators and in particular modulators with primarily antagonistic activities might be useful as medicaments for the treatment of solid tumors (e.g. pancreatic cancer, prostate cancer, breast cancer, colon cancer).

The problem underlying the present invention is to provide compounds which have a AhR-antagonistic activity and can be used in the treatment and/or prophylaxis of AhR-mediated diseases.

Said problem has been solved by a compound according to the following Formulae (I) to (Ill), an enantiomer, diastereomer, tautomer, solvate, N-oxide, prodrug or pharmaceutical acceptable salt thereof

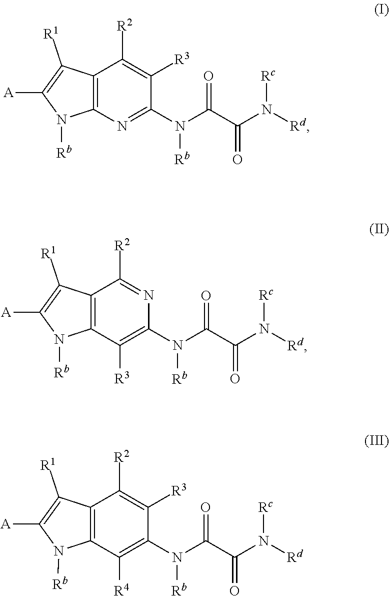

wherein
A represents a 6- to 10-membered mono- or bicyclic aryl or a 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, oxo, C(O)OR^{a}, OC(O)R^{a}, S(O)ₓ—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
   wherein alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, oxo, OH, C₁₋₆-alkyl and halo-C₁₋₆-alkyl;
      R^{a} is independently selected from hydrogen and C₁₋₆-alkyl,
wherein alkyl is unsubstituted or substituted with 1 to 3 substituents independently selected from the group consisting of halogen, halo-C₁₋₃-alkyl, OR^{b} and CN,
or
two R^{a} when taken together with the nitrogen to which they are attached complete a 4- to 8-membered ring containing carbon atoms and optionally containing 1 or 2 heteroatoms independently selected from the group consisting of O, S, and N,
   wherein the 4- to 8-membered ring is unsubstituted or substituted with 1 to 3 substituents independently selected from the group consisting of halogen, halo-C₁₋₃-alkyl, OH, OR^{b} or CN;
      R¹, R², R³ and R⁴ are independently selected from the group consisting of hydrogen, halogen, C₁₋₄-alkyl, halo-C₁₋₃-alkyl, OH, O—C₁₋₃-alkyl and CN;
      R^{b} is independently selected from hydrogen and C₁₋₆-alkyl;
      R^{c} represents hydrogen, C₁₋₆-alkyl,
wherein the C₁₋₆-alkyl is unsubstituted or substituted with 1 to 3 substituents independently selected from halo, CN, oxo, OR^{b}, halo-C₁₋₆-alkyl, C(O)OR^{a}, OC(O)R^{a}, S(O)ₓ—C₁₋₆-alkyl, S(O)(═NR^{b})—C₁₋₆-alkyl, N(R^{a})₂, C(O)N(R^{a})₂, NR^{a}C(O)—C₁₋₆-alkyl, NR^{a}C(O)N(R^{a})₂, S(O)₂N(R^{a})₂, NR^{a}S(O)₂—C₁₋₆-alkyl, NR^{a}S(O)₂N(R^{a})₂, X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), or X—C₀₋₂-alkylene-(5- to 10-membered mono- or bicyclic heteroaryl containing 1 to 4 heteroatoms selected from N, O and S),
   C₃₋₁₀-cycloalkyl,
   3- to 10-membered heterocycloalkyl containing 1 to 4 heteroatoms independently selected from N, O and S,
   6- to 10-membered mono- or bicyclic aryl,
   5- to 10-membered mono- or bicyclic heteroaryl containing 1 to 4 heteroatoms independently selected from N, O and S,
   C₁₋₆-alkylene-C₃₋₁₀-cycloalkyl,
   C₁₋₆-alkylene-C₃₋₁₀-heterocycloalkyl,
   C₁₋₆-alkylene-(6-10-membered mono- or bicyclic aryl), and
   C₁₋₆-alkylene-(5- to 10-membered mono- or bicyclic heteroaryl containing 1 to 4 heteroatoms selected from N, O and S),
wherein alkylene is unsubstituted or substituted with 1 to 3 substituents independently selected from the group consisting of halogen, CN, OH, OR^{b}, oxo, C₁₋₆-alkyl and C₁₋₆-haloalkyl,
wherein cycloalkyl, heterocycloalkyl, aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
   wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo,
or wherein two substituents on the cycloalkyl, heterocycloalkyl, aryl or heteroaryl group together with the atoms they are attached to may form a 5- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, C₁₋₆-alkyl and halo-C₁₋₆-alkyl,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N;
   R^{d} represents a C₁₋₆-alkyl,
wherein the C₁₋₆-alkyl is substituted with 1 to 3 substituents independently selected from halo, CN, oxo, OR^{b}, halo-C₁₋₆-alkyl, C(O)OR^{a}, OC(O)R^{a}, S(O)ₓ—C₁₋₆-alkyl, S(O)(═NR^{b})—C₁₋₆-alkyl, N(R^{a})₂, C(O)N(R^{a})₂, NR^{a}C(O)—C₁₋₆-alkyl, NR^{a}C(O)N(R^{a})₂, S(O)₂N(R^{a})₂, NR^{a}S(O)₂—C₁₋₆-alkyl, NR^{a}S(O)₂N(R^{a})₂, X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), or X—C₀₋₂-alkylene-(5- to 10-membered mono- or bicyclic heteroaryl containing 1 to 4 heteroatoms selected from N, O and S),
   C₃₋₁₀-cycloalkyl,
   3- to 10-membered heterocycloalkyl containing 1 to 4 heteroatoms independently selected from N, O and S,
   6- to 10-membered mono- or bicyclic aryl,
   5- to 10-membered mono- or bicyclic heteroaryl containing 1 to 4 heteroatoms independently selected from N, O and S,
   C₁₋₆-alkylene-C₃₋₁₀-cycloalkyl,
   C₁₋₆-alkylene-C₃₋₁₀-heterocycloalkyl,
   C₁₋₆-alkylene-(6-10-membered mono- or bicyclic aryl), and
   C₁₋₆-alkylene-(5- to 10-membered mono- or bicyclic heteroaryl containing 1 to 4 heteroatoms selected from N, O and S),
wherein alkylene is unsubstituted or substituted with 1 to 3 substituents independently selected from the group consisting of halogen, CN, OH, OR^{b}, oxo, C₁₋₆-alkyl and C₁₋₆-haloalkyl,
wherein cycloalkyl, heterocycloalkyl, aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
   wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo,
or wherein two substituents on the cycloalkyl, heterocycloalkyl, aryl or heteroaryl group together with the atoms they are attached to may form a 5- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, C₁₋₆-alkyl and halo-C₁₋₆-alkyl,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N;
   or
   R^{c} and R^{d} when taken together with the nitrogen to which they are attached complete a 4- to 8-membered saturated or partially unsaturated ring containing carbon atoms and optionally containing 1 or 2 heteroatoms independently selected from the group consisting of O, S, and N,
wherein the ring is unsubstituted or substituted with 1 to 7 substituents independently selected from the group consisting of halogen, C₀₋₄-alkylene-OH, C₀₋₄-alkylene-CN, C₁₋₆-alkyl, C₀₋₄-alkylene-O—C₁₋₆-alkyl, oxo, C₀₋₄-alkylene-C(O)OR^{a}, C₀₋₄-alkylene-OC(O)R^{a}, C₀₋₄-alkylene-S(O)ₓ—C₁₋₆-alkyl, C₀₋₄-alkylene-S(O)(═NR^{b})—C₁₋₆-alkyl, C₀₋₄-alkylene-N(R^{a})₂, C₀₋₄-alkylene-C(O)N(R^{a})₂, C₀₋₄-alkylene-NR^{a}C(O)—C₁₋₆-alkyl, C₀₋₄-alkylene-NR^{a}C(O)N(R^{a})₂, C₀₋₄-alkylene-S(O)₂N(R^{a})₂, C₀₋₄-alkylene-NR^{a}S(O)₂—C₁₋₆-alkyl, C₀₋₄-alkylene-NR^{a}S(O)₂N(R^{a})₂, C₀₋₄-alkylene-C₃₋₁₀-cycloalkyl, C₀₋₄-alkylene-C₃₋₁₀-heterocycloalkyl, C₀₋₄-alkylene-(6-10-membered mono- or bicyclic aryl), C₀₋₄-alkylene-(5- to 10-membered mono- or bicyclic heteroaryl containing 1 to 4 heteroatoms selected from N, O and S), X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), X—C₀₋₂-alkylene-(5- to 10-membered mono- or bicyclic heteroaryl containing 1 to 4 heteroatoms selected from N, O and S), spirocyclic fused C₃₋₇-cycloalkyl and spirocyclic fused 3- to 7-membered heterocycloalkyl containing 1 to 3 heteroatoms independently selected from the group consisting of O, S and N,
   wherein alkylene, alkyl, cycloalkyl, heterocycloalkyl, aryl and heteroaryl are unsubstituted or substituted with 1 to 3 substituents independently selected from the group consisting of halogen, C₁₋₃-alkyl, halo-C₁₋₃-alkyl, OH, CN and oxo,
or wherein two substituents on the 4- to 8-membered ring together with the atoms they are attached to may form a 3- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, oxo, OH, C₁₋₆-alkyl and halo-C₁₋₃-alkyl;
or wherein two substituents on two adjacent carbon atoms on the 4- to 8-membered ring form together with the carbon atoms they are attached to a 5- or 6-membered aromatic ring optionally containing 1 or 2 heteroatoms independently selected from O, N and S,
   wherein the (hetero)aromatic ring is unsubstituted or substituted with 1 to 4 substituents independently selected from the group consisting of halogen, oxo, OH, C₁₋₆-alkyl and halo-C₁₋₃-alkyl;
      X represents —O—, —N(R^{b})—, or —S(O)ₓ—; and
      x is 0, 1 or 2;
      with the proviso that if R^{c} is hydrogen or unsubstituted C₁₋₆-alkyl then R^{d} is not C₃₋₁₀-cycloalkyl, 3- to 10-membered heterocycloalkyl containing 1 to 4 heteroatoms independently selected from N, O and S, 6- to 10-membered mono- or bicyclic aryl or a 5- to 10-membered mono- or bicyclic heteroaryl containing 1 to 4 heteroatoms independently selected from N, O and S.

In a preferred embodiment in combination with any of the above or below embodiments, A is

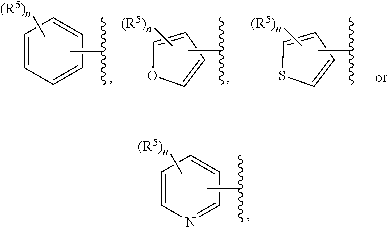

wherein
R⁵ is independently selected from halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂, NR^{a}S(O)₂—C₁₋₆-alkyl and C₃₋₆-cycloalkyl,
wherein alkyl and cycloalkyl are unsubstituted or substituted with 1 to 3 substituents independently selected from the group consisting of halogen, C₁₋₃-alkyl, halo-C₁₋₃-alkyl, OH, CN and oxo;
   R^{a} is independently selected from hydrogen and C₁₋₆-alkyl; and
   n is 0 to 5.

In a more preferred embodiment in combination with any of the above or below embodiments, A is

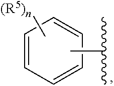

wherein
R⁵ is independently selected from halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂, NR^{a}S(O)₂—C₁₋₆-alkyl and C₃₋₆-cycloalkyl,
wherein alkyl and cycloalkyl are unsubstituted or substituted with 1 to 3 substituents independently selected from the group consisting of halogen, C₁₋₃-alkyl, halo-C₁₋₃-alkyl, OH, CN and oxo;
   R^{a} is independently selected from hydrogen and C₁₋₆-alkyl; and
   n is 0 to 5.

In a most preferred embodiment in combination with any of the above or below embodiments, A is

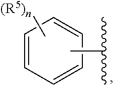

wherein
R⁵ is independently selected from halogen, OH, CN, C₁₋₆-alkyl, O—C₁₋₆-alkyl, and C₃₋₆-cycloalkyl,
wherein alkyl and cycloalkyl are unsubstituted or substituted with 1 to 3 substituents independently selected from the group consisting of halogen, C₁₋₃-alkyl and halo-C₁₋₃-alkyl; and
   n is 0 to 3.

In a further most preferred embodiment in combination with any of the above or below embodiments, A is

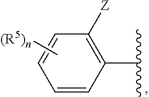

wherein Z is F, Cl, CH₃, CH₂CH₃, CHF₂ or CF₃;
R⁵ is independently selected from halogen and CN; and
n is 0 to 2.

In an utmost preferred embodiment in combination with any of the above or below embodiments, A is

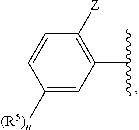

wherein Z is CHF₂ or CF₃;

### R⁵ is C or CN; and

n is 0 or 1.

In a further utmost preferred embodiment in combination with any of the above or below embodiments, A is

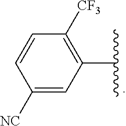 [SMILES: *c1cc(C#N)ccc1C(F)(F)F]

R^{b} is independently selected from hydrogen and C₁₋₆-alkyl.

In a preferred embodiment in combination with any of the above or below embodiments, R^{b} is independently selected from hydrogen and C₁₋₃-alkyl.

In a more preferred embodiment in combination with any of the above or below embodiments, R^{b} is hydrogen.

In a preferred embodiment in combination with any of the above or below embodiments, R¹, R², R³ and R⁴ are independently selected from the group consisting of hydrogen, halogen and C₁₋₃-alkyl.

In a more preferred embodiment in combination with any of the above or below embodiments, R¹, R², R³ and R⁴ are hydrogen.

In a preferred embodiment in combination with any of the above or below embodiments, R^{c} represents hydrogen,

C₁₋₆-alkyl, wherein the C₁₋₆-alkyl is unsubstituted or substituted with 1 to 3 substituents independently selected from halo, CN, oxo, OR^{b}, halo-C₁₋₆-alkyl, C₃₋₆-cycloalkyl,
3- to 6-membered heterocycloalkyl containing 1 to 4 heteroatoms independently selected from N, O and S,
phenyl,
5- to 8-membered mono- or bicyclic heteroaryl containing 1 to 4 heteroatoms independently selected from N, O and S,
C₁₋₄-alkylene-C₃₋₁₀-cycloalkyl,
C₁₋₄-alkylene-C₃₋₆-heterocycloalkyl,
C₁₋₄-alkylene-phenyl, and
C₁₋₄-alkylene-(5- to 8-membered mono- or bicyclic heteroaryl containing 1 to 4 heteroatoms selected from N, O and S),
wherein alkylene is unsubstituted or substituted with 1 to 3 C₁₋₆-alkyl groups,
wherein cycloalkyl, heterocycloalkyl, phenyl and heteroaryl are unsubstituted or substituted with 1 to 7 substituents independently selected from the group consisting of halogen, OH, CN and O—C₁₋₆-alkyl,
   wherein the alkyl is unsubstituted or substituted with 1 to 3 substituents independently selected from the group consisting of halogen, C₁₋₃-alkyl and OH,
or wherein two substituents on the cycloalkyl, heterocycloalkyl, phenyl or heteroaryl group together with the atoms they are attached to may form a 5- to 7-membered saturated or partially unsaturated carbocyclic ring or heterocyclic ring containing 1 to 3 heteroatoms independently selected from O, N and S
   wherein the carbocyclic or heterocyclic ring is unsubstituted or substituted with 1 to 5 substituents independently selected from the group consisting of halogen, C₁₋₆-alkyl and halo-C₁₋₆-alkyl,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N.

In a more preferred embodiment in combination with any of the above or below embodiments, R^{c} represents hydrogen or C₁₋₆-alkyl, wherein the C₁₋₆-alkyl is unsubstituted or substituted with 1 to 3 substituents independently selected from halo, CN, oxo, OR^{b} and halo-C₁₋₆-alkyl.

In a most preferred embodiment in combination with any of the above or below embodiments, R^{c} is C₁₋₆-alkyl, wherein the C₁₋₆-alkyl is unsubstituted or substituted with 1 to 3 substituents independently selected from halo, CN, oxo, OR^{b} and halo-C₁₋₆-alkyl.

In a preferred embodiment in combination with any of the above or below embodiments, R^{d} represents a C₁₋₆-alkyl, wherein the C₁₋₆-alkyl is substituted with 1 to 3 substituents independently selected from halo, CN, oxo, OR^{b}, halo-C₁₋₆-alkyl, C(O)OR^{a}, OC(O)R^{a}, S(O)ₓ—C₁₋₆-alkyl, S(O)(═NR^{b})—C₁₋₆-alkyl, N(R^{a})₂, C(O)N(R^{a})₂, NR^{a}C(O)—C₁₋₆-alkyl, NR^{a}C(O)N(R^{a})₂, S(O)₂N(R^{a})₂, NR^{a}S(O)₂—C₁₋₆-alkyl, NR^{a}S(O)₂N(R^{a})₂, X—C₀₋₂-alkylene-C₃₋₆-cycloalkyl, X—C₀₋₂-alkylene-C₃₋₈-heterocycloalkyl, X—C₀₋₂-alkylene-phenyl, or X—C₀₋₂-alkylene-(5- to 8-membered mono- or bicyclic heteroaryl containing 1 to 4 heteroatoms selected from N, O and S),

C₃₋₆-cycloalkyl,
3- to 8-membered heterocycloalkyl containing 1 to 4 heteroatoms independently selected from N, O and S,
phenyl,
5- to 8-membered mono- or bicyclic heteroaryl containing 1 to 4 heteroatoms independently selected from N, O and S,
C₁₋₄-alkylene-C₃₋₆-cycloalkyl,
C₁₋₄-alkylene-C₃₋₈-heterocycloalkyl,
C₁₋₄-alkylene-phenyl, and
C₁₋₄-alkylene-(5- to 8-membered mono- or bicyclic heteroaryl containing 1 to 4 heteroatoms selected from N, O and S),
wherein alkylene is unsubstituted or substituted with 1 to 3 C₁₋₆-alkyl groups,
wherein cycloalkyl, heterocycloalkyl, phenyl and heteroaryl are unsubstituted or substituted with 1 to 7 substituents independently selected from the group consisting of halogen, OH, CN and O—C₁₋₆-alkyl,
   wherein the alkyl is unsubstituted or substituted with 1 to 3 substituents independently selected from the group consisting of halogen, C₁₋₃-alkyl and OH,
or wherein two substituents on the cycloalkyl, heterocycloalkyl, phenyl or heteroaryl group together with the atoms they are attached to may form a 5- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, C₁₋₆-alkyl and halo-C₁₋₆-alkyl,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N.

In a more preferred embodiment in combination with any of the above or below embodiments, R^{d} represents a C₁₋₆-alkyl, wherein the C₁₋₆-alkyl is substituted with 1 to 3 substituents independently selected from halo, CN, oxo, OR^{b}, halo-C₁₋₆-alkyl, X—C₀₋₂-alkylene-C₃₋₆-cycloalkyl, X—C₀₋₂-alkylene-C₃₋₈-heterocycloalkyl, X—C₀₋₂-alkylene-phenyl, or X—C₀₋₂-alkylene-(5- to 8-membered mono- or bicyclic heteroaryl containing 1 to 4 heteroatoms selected from N, O and S),

C₃₋₆-cycloalkyl,
3- to 8-membered heterocycloalkyl containing 1 to 4 heteroatoms independently selected from N, O and S,
phenyl,
5- to 8-membered mono- or bicyclic heteroaryl containing 1 to 4 heteroatoms independently selected from N, O and S,
C₁₋₄-alkylene-C₃₋₆-cycloalkyl,
C₁₋₄-alkylene-C₃₋₁₀-heterocycloalkyl,
C₁₋₄-alkylene-phenyl, and
C₁₋₄-alkylene-(5- to 8-membered mono- or bicyclic heteroaryl containing 1 to 4 heteroatoms selected from N, O and S),
wherein alkylene is unsubstituted or substituted with 1 to 3 C₁₋₃-alkyl groups,
wherein cycloalkyl, heterocycloalkyl, phenyl and heteroaryl are unsubstituted or substituted with 1 to 7 substituents independently selected from the group consisting of halogen, OH, CN and O—C₁₋₆-alkyl,
or wherein two substituents on the cycloalkyl, heterocycloalkyl, phenyl or heteroaryl group together with the atoms they are attached to may form a 5- to 7-membered saturated or partially unsaturated carbocyclic ring or heterocyclic ring containing 1 to 3 heteroatoms independently selected from O, N and S,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N.

In an even more preferred embodiment in combination with any of the above or below embodiments, R^{d} represents a C₁₋₆-alkyl, wherein the C₁₋₆-alkyl is substituted with 1 to 3 substituents independently selected from halo, CN, oxo, OR^{b} and halo-C₁₋₆-alkyl,

C₁₋₄-alkylene-C₃₋₆-cycloalkyl,
C₁₋₄-alkylene-C₃₋₁₀-heterocycloalkyl,
C₁₋₄-alkylene-phenyl, and
C₁₋₄-alkylene-(5- to 8-membered mono- or bicyclic heteroaryl containing 1 to 4 heteroatoms selected from N, O and S),
wherein alkylene is unsubstituted or substituted with 1 to 3 C₁₋₃-alkyl groups,
wherein cycloalkyl, heterocycloalkyl, phenyl and heteroaryl are unsubstituted or substituted with 1 to 7 substituents independently selected from the group consisting of halogen, OH, CN and O—C₁₋₆-alkyl,
   or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N.

In a preferred embodiment in combination with any of the above or below embodiments,

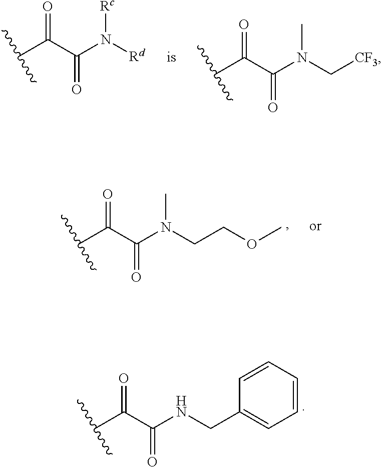

In a preferred embodiment in combination with any of the above and below embodiments, R^{c} and R^{d} when taken together with the nitrogen to which they are attached complete a 4- to 8-membered saturated or partially unsaturated ring containing carbon atoms and optionally containing 1 or 2 heteroatoms independently selected from the group consisting of O, S, and N,
wherein the ring is unsubstituted or substituted with 1 to 5 substituents independently selected from the group consisting of halogen, C₀₋₄-alkylene-OH, C₀₋₄-alkylene-CN, C₁₋₆-alkyl, C₀₋₄-alkylene-O—C₁₋₆-alkyl, oxo, C₀₋₄-alkylene-C(O)OR^{a}, C₀₋₄-alkylene-OC(O)R^{a}, C₀₋₄-alkylene-S(O)ₓ—C₁₋₆-alkyl, C₀₋₄-alkylene-S(O)(═NR^{b})—C₁₋₆-alkyl, C₀₋₄-alkylene-N(R^{a})₂, C₀₋₄-alkylene-C(O)N(R^{a})₂, C₀₋₄-alkylene-NR^{a}C(O)—C₁₋₆-alkyl, C₀₋₆-alkylene-NR^{a}C(O)N(R^{a})₂, C₀₋₄-alkylene-S(O)₂N(R^{a})₂, C₀₋₄-alkylene-NR^{a}S(O)₂—C₁₋₆-alkyl, C₀₋₄-alkylene-NR^{a}S(O)₂N(R^{a})₂, C₀₋₄-alkylene-C₃₋₁₀-cycloalkyl, C₀₋₄-alkylene-C₃₋₁₀-heterocycloalkyl, C₀₋₄-alkylene-(6-10-membered mono- or bicyclic aryl), C₀₋₄-alkylene-(5- to 10-membered mono- or bicyclic heteroaryl containing 1 to 4 heteroatoms selected from N, O and S), X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), X—C₀₋₂-alkylene-(5- to 10-membered mono- or bicyclic heteroaryl containing 1 to 4 heteroatoms selected from N, O and S), spirocyclic fused C₃₋₇-cycloalkyl and spirocyclic fused 3- to 7-membered heterocycloalkyl containing 1 to 3 heteroatoms independently selected from the group consisting of O, S and N,
   wherein alkylene, alkyl, cycloalkyl, heterocycloalkyl, aryl and heteroaryl are unsubstituted or substituted with 1 to 2 substituents independently selected from the group consisting of halogen and C₁₋₃-alkyl,
or wherein two substituents on the 4- to 8-membered ring together with the atoms they are attached to may form a 3- to 7-membered saturated or partially unsaturated carbocyclic ring or heterocyclic ring containing 1 to 3 heteroatoms independently selected from O, N and S,
   wherein the carbocyclic or heterocyclic ring is unsubstituted or substituted with 1 to 5 substituents independently selected from the group consisting of halogen, oxo, OH, C₁₋₆-alkyl and halo-C₁₋₃-alkyl,
or wherein two substituents on two adjacent carbon atoms on the 4- to 8-membered ring form together with the carbon atoms they are attached to a 5- or 6-membered aromatic ring optionally containing 1 or 2 heteroatoms independently selected from O, N and S,
   wherein the (hetero)aromatic ring is unsubstituted or substituted with 1 to 4 substituents independently selected from the group consisting of halogen, oxo, OH, C₁₋₆-alkyl and halo-C₁₋₃-alkyl.

In an even more preferred embodiment in combination with any of the above and below embodiments, R^{c} and R^{d} when taken together with the nitrogen to which they are attached complete a 4- to 6-membered saturated or partially unsaturated ring containing carbon atoms and optionally containing 1 or 2 heteroatoms independently selected from the group consisting of O, S, and N,
wherein the ring is unsubstituted or substituted with 1 to 3 substituents independently selected from the group consisting of halogen, C₀₋₄-alkylene-OH, C₀₋₄-alkylene-CN, C₁₋₆-alkyl and C₀₋₄-alkylene-O—C₁₋₆-alkyl,
   wherein alkylene and alkyl are unsubstituted or substituted with 1 or 2 substituents independently selected from the group consisting of halogen, OH and CN.

In a most preferred embodiment in combination with any of the above and below embodiments,

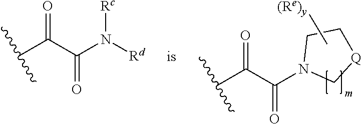

wherein R^{e} is independently selected from the group consisting of halogen, CN, OH, O—C₁₋₆-alkyl, C₁₋₆-alkyl, spirocyclic fused C₃₋₇-cycloalkyl and spirocyclic fused 3- to 7-membered heterocycloalkyl containing 1 heteroatom selected from the group consisting of O, S and N;
Q is CH₂, CHR^{e}, O, NH, N—C₁₋₆-alkyl or S;
m is 0, 1 or 2, and
y is 0, 1 or 2.

In an utmost preferred embodiment in combination with any of the above and below embodiments, R^{c} and R^{d} when taken together with the nitrogen to which they are attached complete a 4- to 6-membered saturated or partially unsaturated ring containing carbon atoms and optionally containing 1 additional heteroatom selected from the group consisting of O, S, and N,
wherein the ring is unsubstituted or substituted with 1 substituent selected from the group consisting of halogen and C₁₋₆-alkyl.

In a further utmost preferred embodiment in combination with any of the above and below embodiments,

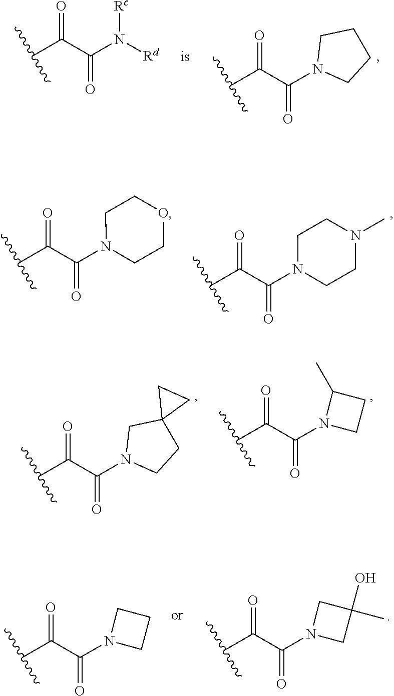

In an equally utmost preferred embodiment in combination with any of the above and below embodiments,

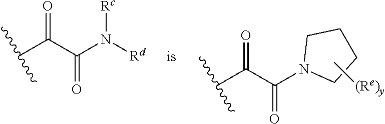

wherein R^{e} is halogen, CN, OH, O—C₁₋₆-alkyl, C₁₋₆-alkyl, spirocyclic fused C₃₋₇-cycloalkyl and spirocyclic fused 3- to 7-membered heterocycloalkyl containing 1 heteroatom selected from the group consisting of O, S and N, and
y is 0, 1 or 2.

In yet another equally most preferred embodiment in combination with any of the above and below embodiments,

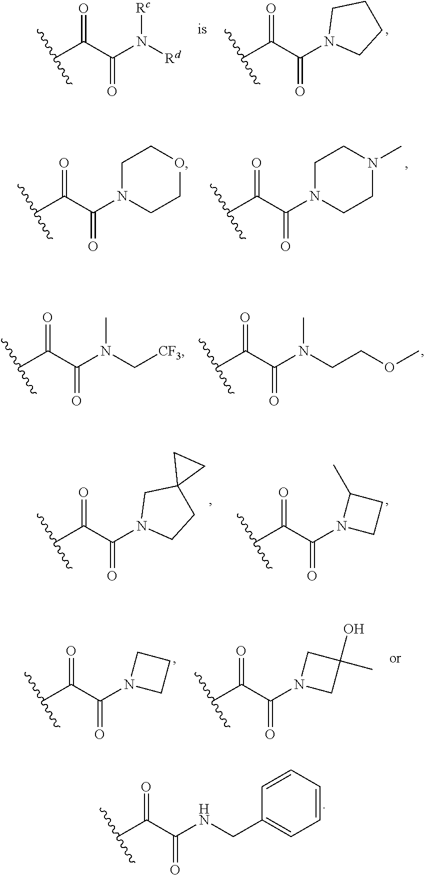

In an utmost preferred embodiment in combination with any of the above and below embodiments,

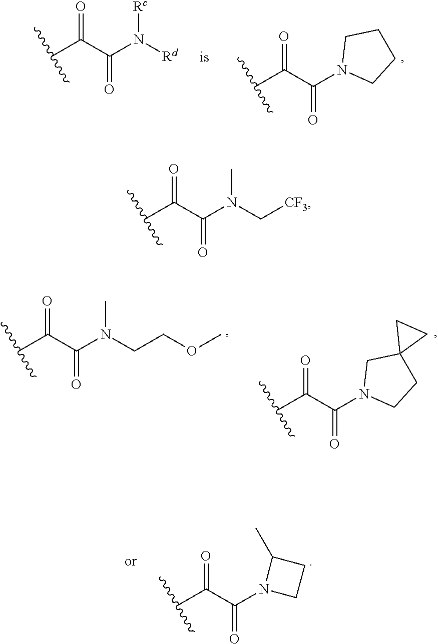

In a preferred embodiment in combination with any of the above and below embodiments, the compound is represented by Formulae (I) or (II).

In a more preferred embodiment in combination with any of the above and below embodiments, the compound is represented by Formula (I).

In an equally more preferred embodiment in combination with any of the above and below embodiments, the compound is represented by Formula (II).

In a preferred embodiment in combination with any of the above and below embodiments, the compound is selected from the group consisting of

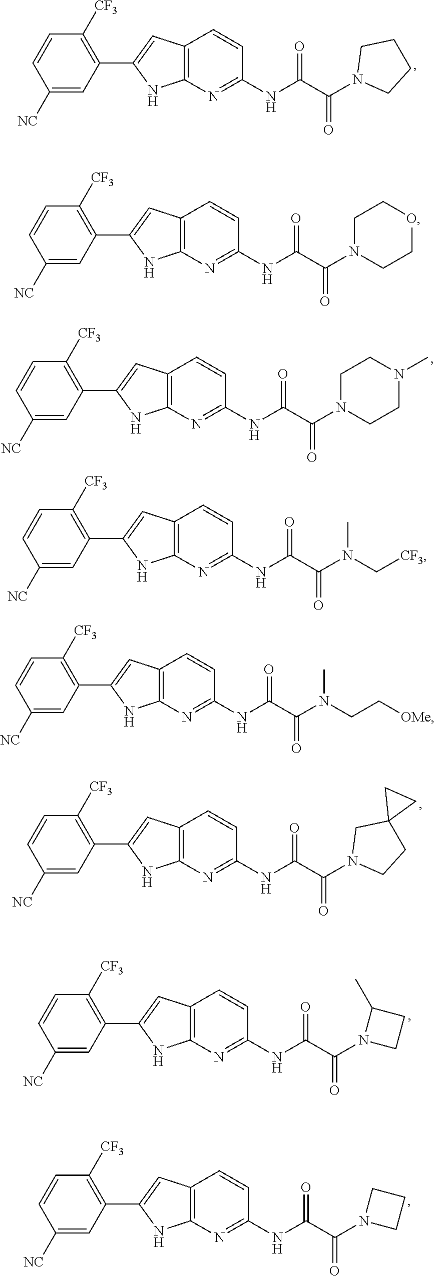

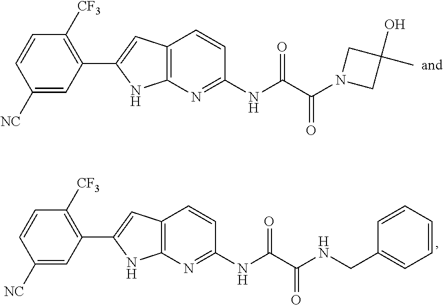 [SMILES: CC1(O)CN(C(=O)C(=O)Nc2ccc3cc(-c4cc(C#N)ccc4C(F)(F)F)[nH]c3n2)C1 ; N#Cc1ccc(C(F)(F)F)c(-c2cc3ccc(NC(=O)C(=O)NCc4ccccc4)nc3[nH]2)c1]

and an enantiomer, diastereomer, tautomer, solvate, N-oxide, prodrug or pharmaceutical acceptable salt thereof.

In another embodiment, the present invention is directed to a pharmaceutical composition comprising the compound according to Formulae (I) to (Ill) and a physiologically acceptable excipient.

In another embodiment, the present invention is directed to the compound according to Formulae (I) to (III) for use as a medicament.

In another embodiment, the present invention is directed to the compound according to Formulae (I) to (III) or a pharmaceutical composition containing same and a physiologically acceptable excipient for use in the prophylaxis and/or treatment of a disease or condition mediated by aryl hydrocarbon receptor (AhR).

In another embodiment in combination with any of the above or below embodiments, the disease or condition mediated by aryl hydrocarbon receptor (AhR) is cancer.

In another embodiment in combination with any of the above or below embodiments, the compound according to Formulae (I) to (III) is administered with one or more therapeutic agents for cancer selected from the group consisting of PD-1 agent, PD-L1 agent, CTLA-4 agent, IDO1 inhibitor, chemotherapeutic agent, anticancer vaccine, Toll like receptor agonist, oncolytic virus, STING agonist and cytokine therapy, or wherein the compound is administered under irradiation therapy.

In the context of the present invention “C₁₋₆-alkyl” means a saturated alkyl chain having 1, 2, 3, 4, 5 or 6 carbon atoms which may be straight chained or branched. In the context of the present invention, any subgroup falling within the term “C₁₋₆-alkyl” is also encompassed such as a C₁₋₃-alkyl, C₂-s-alkyl or C₃₋₆-alkyl group. Examples of the C₁₋₆-alkyl group include methyl, ethyl, propyl, isopropyl, n-butyl, isobutyl, tert-butyl, n-pentyl, isopentyl, neopentyl, and hexyl.

A “C₀₋₂-alkylene” means that the respective group is divalent and connects the attached residue with the remaining part of the molecule. Moreover, in the context of the present invention, “C₀-alkylene” is meant to represent a bond, whereas C₁-alkylene means a methylene linker, C₂-alkylene means an ethylene linker or a methyl-substituted methylene linker and so on. In the context of the present invention, a C₀₋₂-alkylene preferably represents a bond or a methylene group.

The term “O—C₁₋₆-alkyl” means that the alkyl chain is connected via an oxygen atom with the remainder of the molecule. Similarly, the term “S(O)ₓ—C₁₋₆-alkyl” defines an alkyl chain which is connected via the S(O)ₓ-group with the remainder of the molecule and so on.

The term “halo-C₁₋₆-alkyl” means that one or more hydrogen atoms in the alkyl chain are replaced by a halogen. A preferred example thereof is CF₃.

A C₃₋₁₀-cycloalkyl group means a saturated or partially unsaturated mono- or bicyclic ring system comprising 3, 4, 5, 6, 7, 8, 9, or 10 carbon atoms. Examples include cyclopropyl, cyclobutyl, cyclopentyl, cyclohexyl, cyclohexenyl, cycloheptyl, cycloheptenyl, cyclooctyl, cyclononyl, cyclodecyl and decalin. In a similar manner, a C₃₋₇-cycloalkyl group means a saturated or partially unsaturated mono- or bicyclic ring system comprising 3, 4, 5, 6 or 7 carbon atoms.

A 3- to 10-membered heterocycloalkyl group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O, S, SO and SO₂. Examples thereof include epoxidyl, oxetanyl, pyrrolidinyl, tetrahydrofuranyl, piperidinyl, piperazinyl, tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom.

Furthermore, two substituents on a cycloalkyl or heterocycloalkyl group may form a saturated or partially unsaturated carbocyclic or heterocyclic ring whereby the newly formed ring is connected in a spirocyclic manner as shown below which is also designated as “spirocyclic fused” ring:

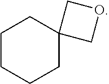

A 5-10-membered mono- or bicyclic heteroaromatic ring system (within the application also referred to as heteroaryl) containing up to 6 heteroatoms means a monocyclic heteroaromatic ring such as pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include, benzimidazolyl, benzisoxazolyl, benzodioxanyl, benzofuranyl, benzoxazolyl, imidazo[1,2-a]pyridinyl, imidazo[1,2-c]pyrimidinyl, indazolyl, indolizinyl, indolyl, isoquinolinyl, pyrazolo[1.5-a]pyridinyl, pyrrolo[2,3-b]pyridinyl, pyrrolo[3,2-c]pyridinyl, pyrrolo[2,3-d]thiazolyl, quinolinyl, quinoxalinyl, pyrazolo[1,5-a]pyrimidinyl, thiazolo[4,5-b]pyridinyl and [1,2,4]triazolo[1,5-a]pyrimidinyl.

The nitrogen or sulphur atom of the heteroaryl system may also be optionally oxidized to the corresponding N-oxide, S-oxide or S,S-dioxide. If not stated otherwise, the heteroaryl system can be connected via a carbon or nitrogen atom. Examples for N-linked heterocycles are

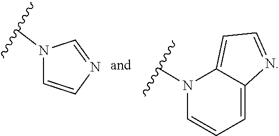 [SMILES: *n1ccnc1 ; *n1cccc2nccc1-2]

Moreover, where not explicitly defined, heteroaryl contains 1 to 6 heteroatoms independently selected from the group consisting of N, O and S.

A 6-10-membered mono- or bicyclic aromatic ring system (within the application also referred to as aryl) means an aromatic carbon cycle such as phenyl or naphthyl.

The term “halogen” comprises the specific halogen atoms fluorine, bromine, chlorine and iodine.

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, fluorine and chlorine, such as, but not limited to ²H (deuterium, D), ³H (tritium), ¹¹C, ¹³C, ¹⁴C, ¹⁵N, ¹⁸F, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The compounds of the present invention can be in the form of a prodrug compound. “Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods.

Other examples of the prodrug are compounds, wherein the carboxylate in a compound of the present invention is, for example, converted into an alkyl-, aryl-, choline-, amino, acyloxymethylester, linolenoylester.

Metabolites of compounds of the present invention are also within the scope of the present invention.

Where tautomerism, like e.g. keto-enol tautomerism, of compounds of the present invention or their prodrugs may occur, the individual forms, like e.g. the keto and enol form, are each within the scope of the invention as well as their mixtures in any ratio. Same applies for stereoisomers, like e.g. enantiomers, cis/trans isomers, conformers and the like.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials. Another way to obtain pure enantiomers from racemic mixtures would use enantioselective crystallization with chiral counterions.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further the compounds of the present invention may be present in the form of solvates, such as those which include as solvate water, or pharmaceutically acceptable solvates, such as alcohols, in particular ethanol.

Furthermore, the present invention provides pharmaceutical compositions comprising at least one compound of the present invention, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier.

The pharmaceutical composition of the present invention may additionally comprise one or more other compounds as active ingredients like a prodrug compound or other nuclear receptor modulators.

Specifically, the compound of the present invention can be administered with one or more therapeutic agents for cancer selected from the group consisting of PD-1 agent, PD-L1 agent, CTLA-4 agent, IDO1 inhibitor, chemotherapeutic agent, anticancer vaccine, Toll like receptor agonist, oncolytic virus, STING agonist and cytokine therapy, or the compound is administered under irradiation therapy.

Examples of PD-1 agents include, but are not limited to, Pembrolizumab and Nivolumab.

Examples of PD-L1 agents include, but are not limited to, Atezolizumab, Avelumab and Durvalumab.

Examples of CTLA-4 agents include, but are not limited to, Ipilimumab.

Examples of IDO1 inhibitors include, but are not limited to, Epacadostat, Navoximod and BMS-986205.

Examples of chemotherapeutic agents include, but are not limited to, Cyclophosphamide, Busulfan, Carmustin, Temozolimide, Procarbazin, Trabectedin, Cisplatin, Carboplatin, Methotrexat, Pemetrexed, 6-Mercatopurine, 6-Thioguanine, Cladibine, Clofarabine, Nelarabine, Pentostatine, 5-Fluorouracil, Cytarabine, Gemcitabine, Azacitidine, Vincristine, Vinblastine, Vindesine, Paclitaxel, Docetaxel, Cabazitaxel, Ixabepilone, Eribulin, Estramustine phosphate, Topotecan, Irinotecan, Etoposide, Teniposide, Dactinomycin, Bleomycin, Doxorubicin, Daunorubicin, Epirubicin, Idarubicin, Mitoxantron, all-trans retinoic acid, Bexarotene, As₂O₃, Imatinib, Nilotinib, Dasatinib, Bosutinib, Ponatinib, Erlotinib, Gefitinib, Afatinib, Osimertinib, Lapatinib, Crizotinib, Ceritinib, Axitinib, Cabozantinib, Lanvatinib, Nintedanib, Pazopanib, Regorafenib, Sorafenib, Sunitinib, Ruxolitinib, Dovitinib, Ibrutinib, Idelalisib, Vemurafenib, Dabrafenib, Trametinib, Cobimetinib, Palbociclib, Temsirolismus, Everolimus, Bortezomib, Carfilzomib, Vismodegib, Panobinostat, Olaparib, Venetoclax, Rituximab, Trastuzumab, Pertuzumab, Cetuximab, Panitumumab, Necitumumab, Bevacizumab, Ramucirumab, Olaratumab, Mifamurtide, Elotuzumab, Catumaxomab, Blinatumomab, Rituximab, Daratumumab, Alemtuzumab, Prednisone, Buserelin, Goserelin, Leuprorelin, Histrelin, Triptorelin, Degarelix, Abarelix, Flutamide, Bicalutmide, Enzalutamide, Arbiraterone, Tamoxifen, Toremifen, Exemestane, Letrozole, Anastrozole, Fulvestrant, Thalidomide, Lenalidomide and Pomalidomide.

Examples of anticancer vaccines include, but are not limited to, Hepa-VAC-101 and Sipuleucel-T.

Examples of Toll like receptor agonists include, but are not limited to Imiquimod, Resiquimod, monophosphoryl lipid A, BCG, CpG ODNs and Motolimod.

Examples of oncolytic viruses include, but are not limited to H101, Talimogene laherparepvec.

Examples of STING agonists include, but are not limited to, ADU-S100 and MK-1454.

Examples of cytokine therapy include, but are not limited to IL-2, GM-CSF, IL-12 and IL-10.

Examples of other Immune-Oncology therapeutics that can be used in combination with the compounds of the present invention include, but are not limited to Chimeric antigen receptor, or CAR T-cell therapy, such as Tisagenlecleucel, Axicabtagen Ciloleucel and immune response modifying enzymes such as Asparaginase or Kynureninase.

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions, or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatine; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavouring such as cherry or orange flavour.

The compounds used in the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous, intramuscular and subcutaneous), ocular (ophthalmic), pulmonary (nasal or buccal inhalation), nasal and the like may be employed. Dosage forms include tablets, troches, dispersions, suspensions, solutions, capsules, creams, ointments, aerosols, and the like. Preferably, compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing AhR-mediated conditions for which compounds of Formula (I) are indicated, generally satisfactory results are obtained when the compounds are administered at a daily dosage of from about 0.1 mg to about 100 mg per kilogram of mammal body weight, preferably given as a single daily dose or in divided doses two to six times a day, or in sustained release form. For most large mammals, the total daily dosage is from about 1 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

### ABBREVIATIONS

Herein and throughout the application, the following abbreviations may be used.
ACN acetonitrile
aq. aqueous
br broad
d doublet
DCM dichloromethane
DMA N,N-dimethylacetamide
DMF N,N-dimethylformamide
DMSO dimethylsulfoxide
EDC N-(3-dimethylaminopropyl)-N′-ethylcarbodiimide hydrochloride
EtOAc ethyl acetate
Et₂O diethylether
eq. equivalents
HOBt 1-hydroxybenzotriazole
RP-HPLC reversed phase high performance liquid chromatography
m multiplet
rpm rounds per minute
rt room temperature
s singlet
t-BuOK potassium tert-butoxide

### General Schemes

The compounds of the present invention can be prepared by a combination of methods known in the art including the procedures described in scheme 1 below. The following reaction schemes are only meant to represent examples of the invention and are in no way meant to be a limit of the invention.

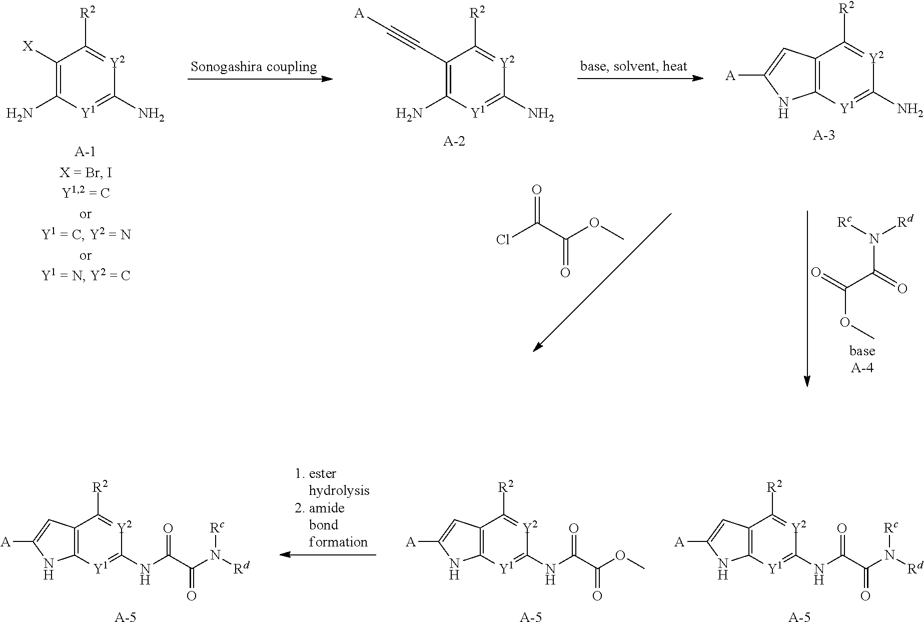

Diaminopyridines or diaminobenzenes A-1 with a substitution pattern as indicated in scheme 1 can be substituted by terminal acetylenes under Pd-mediated cross-coupling reaction conditions like e.g. in a Sonogashira reaction. The resulting acetylene substituted diaminopyridines or diaminobenzenes A-2 can be cyclized to aminoindole or aminoazaindole derivatives A-3 upon heating in the presence of an appropriate base in an appropriate solvent. The introduction of an oxalamide-containing residue on the amino group of A-3 leads to compounds A-5 of the present invention. This can either be achieved in one step by treating A-3 with esters A-4 in the presence of an appropriate base at low temperature or in a two step sequence whereby A-3 is first reacted with methyl 2-chloro-2-oxoacetate and after ester hydrolysis of intermediate A-6 a final amide bond formation is performed to afford compounds A-5 of the present invention.

### Intermediate 1: 3-(6-Amino-1H-pyrrolo[2,3-b]pyridin-2-yl)-4-(trifluoromethyl)benzonitrile (Int 1)

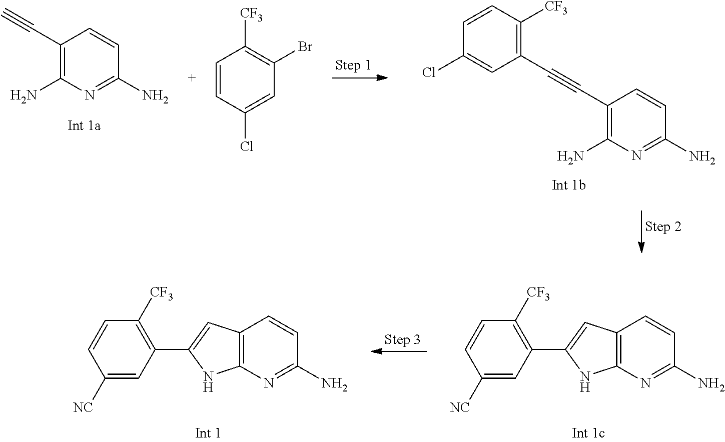

### Step 1: 3-((5-Chloro-2-(trifluoromethyl)phenyl)ethynyl)pyridine-2,6-diamine (Int 1b)

A mixture of 3-ethynyl-pyridine-2,6-diamine (Int 1a) (2.14 g, 16.0 mmol, 1.0 eq), tetrakis(triphenylphosphine)palladium(0) (1.98 g, 1.7 mmol, 0.11 eq.), copper (I) iodide (431 mg, 2.26 mmol, 0.14 eq.) and 2-bromo-4-chlorobenzotrifluoride (3.5 mL, 24.1 mmol, 1.5 eq.) in degassed isopropylamine (85.6 mL, 16.0 mmol, 1.0 eq.) was stirred and heated under an atmosphere of nitrogen at 80° C. for 1 h. The mixture was cooled to rt, and partitioned between 600 mL EtOAc and 300 mL water. The separated aq. phase was extracted again with 150 mL EtOAc and the combined organic layer was dried over Na₂SO₄, filtered and concentrated. The crude product was purified by flash chromatography on silicagel using cyclohexane and EtOAc as eluents (25 to 75% EtOAc within 9 column volumes). The pooled product fractions were concentrated to dryness under reduced pressure. The obtained yellow solid was triturated with cyclohexane (200 mL) by stirring vigorously (800 rpm) at 40° C. for 1 h. The solid was filtered off and dried under air stream (10 min) and under vacuum (25 mbar) for 1 h to afford the title compound as a yellow powder. ¹H-NMR (300 MHz, CDCl₃): δ ppm 7.63-7.53 (m, 2H), 7.38 (d, J=8.1 Hz, 1H), 7.32 (d, J=8.4 Hz, 1H), 5.88 (d, J=8.1 Hz, 1H), 4.93 (s br, 2H), 4.46 (s br, 2H). MS (ESI): m/z 312.2 [M+H]+.

### Step 2: 2-(5-Chloro-2-(trifluoromethyl)phenyl)-1H-pyrrolo[2,3-b]pyridin-6-amine (Int 1c)

To a solution of 3-((5-chloro-2-(trifluoromethyl)phenyl)ethynyl)pyridine-2,6-diamine (Int 1b) (4.3 g, 13.8 mmol, 1.0 eq.) in DMSO (170 mL), under nitrogen was added t-BuOK (1.9 g, 17.0 mmol, 1.2 eq.) at rt. A deep red colour formed and the mixture was stirred and heated to 80° C. for 16.5 h. The mixture was cooled to rt and neutralized by addition of 1N aq. HCl (18 mL, final pH ca. 5.5). The solvent was removed under reduced pressure and the residue was partitioned between EtOAc (300 mL) and sat. aq. NaHCO₃ (200 mL). The organic phase was washed with brine, dried over Na₂SO₄, filtered and concentrated to dryness. The obtained dark brown residue was purified by flash chromatography on silicagel using cyclohexane and EtOAc as eluents (20 to 80% EtOAc within 10 column volumes). The pooled product fractions were concentrated to dryness to afford a brown powder. The product was recrystallized from Et₂O and cyclohexane to afford the title compound as a light brown crystalline powder. ¹H-NMR (300 MHz, DMSO-d₆): δ ppm 11.29 (s, 1H), 7.84 (d, J=8.4 Hz, 1H), 7.71 (s, 1H), 7.60 (d, J=8.3 Hz, 2H), 6.41 (s, 1H), 6.31 (d, J=8.4 Hz, 1H), 5.84 (s br, 2H). MS (ESI): m/z 312.2 [M+H]⁺.

### Step 3: 3-(6-Amino-1H-pyrrolo[2,3-b]pyridin-2-yl)-4-(trifluoromethyl)benzonitrile (Int 1)

A mixture of [1,1′-binaphthalen]-2-yldi-tert-butylphosphane (1.06 g, 2.7 mmol, 0.45 eq.), palladium(II) trifluoroacetate (465 mg, 1.4 mmol, 0.24 eq.), zinc cyanide (1.04 g, 8.9 mmol, 1.5 eq.), and zinc powder (55 mg, 0.8 mmol, 0.14 eq.) in degassed DMA (11 mL) was stirred and heated to 95° C. for 5 min under an atmosphere of nitrogen. A dark violet colour in a black suspension formed. Then a solution of 2-(5-chloro-2-(trifluoromethyl)phenyl)-1H-pyrrolo[2,3-b]pyridin-6-amine (Int 1c) (1.84 g, 5.9 mmol) and acetic acid (433 μL, 7.5 mmol, 1.3 eq.) in degassed DMA (11 mL) was added within 12 minutes. Stirring was continued at 95° C. for 70 min. After cooling to rt the mixture was diluted with DCM (200 mL) and filtered over cellite. A yellow solution was obtained which was washed with sat. aq. NaHCO₃ (80 mL), dried over Na₂SO₄ and filtered. The obtained organic phase was concentrated to dryness under vacuum. The residue was purified by flash chromatography on silicagel using cyclohexane and acetone as eluents (10 to 65% EtOAc within 12 column volumes). The combined product fractions were concentrated to dryness to afford the title compound as a yellow powder. ¹H-NMR (300 MHz, DMSO-d₆): δ ppm 11.31 (s, 1H), 8.13 (s, 1H), 8.07-7.93 (m, 2H), 7.61 (d, J=8.4 Hz, 1H), 6.45 (s, 1H), 6.31 (d, J=8.4 Hz, 1H), 5.88 (s br, 2H). MS (ESI): m/z 308.2 [M+H]⁺.

### Intermediate 2: Methyl 2-morpholino-2-oxoacetate (Int 2)

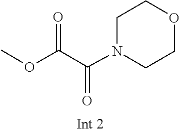

To a stirred solution of morpholine (137 mg, 1.6 mmol) and triethylamine (160 mg, 1.6 mmol) in DCM (1.6 mL) at 0° C. was added methyl 2-chloro-2-oxoacetate (200 mg, 1.6 mmol). The mixture was stirred for 1 h at rt, poured on sat. aq. NaHCO₃ (15 mL) and extracted with DCM (3×15 mL). The combined organic layers were washed with brine, dried over MgSO₄, filtered and concentrated under reduced pressure to afford the title compound as a slightly yellow liquid. ¹H-NMR (300 MHz, DMSO-ds): δ ppm 3.81 (s, 3H), 3.94-3.54 (m, 4H), 3.53-3.45 (m, 2H), 3.41-3.35 (m, 2H). MS (ESI): m/z 174.3 [M+H]⁺.

### Intermediate 2/1: Methyl 2-oxo-2-(pyrrolidin-1-yl)acetate (Int 2/1)

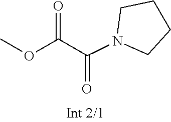

The title compound was prepared similar as described for Intermediate 2. ¹H NMR (300 MHz, DMSO-ds): δ ppm 3.79 (s, 3H), 3.50-3.42 (m, 2H), 3.38-3.31 (m, 2H), 1.92-1.76 (m, 4H). MS (ESI): m/z 158.3 [M+H]⁺.

### Intermediate 2/2: Methyl 2-(4-methylpiperazin-1-yl)-2-oxoacetate (Int 2/2)

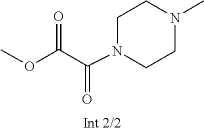

The title compound was prepared similar as described for Intermediate 2. ¹H-NMR (300 MHz, CD₃OD): δ ppm 3.87 (s, 3H), 3.67-3.58 (m, 2H), 3.51-3.44 (m, 2H), 2.52-2.43 (m, 4H). 2.33 (s, 3H). MS (ESI): m/z 187.3 [M+H]⁺.

### Intermediate 2/3: Methyl 2-(methyl(2,2,2-trifluoroethyl)amino)-2-oxoacetate (Int 2/3)

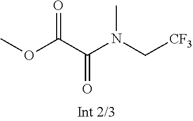

The title compound was prepared similar as described for Intermediate 2. MS (ESI): m/z 200.2 [M+H]⁺.

### Intermediate 2/4: Methyl 2-((2-methoxyethyl)(methyl)amino)-2-oxoacetate (Int 2/4)

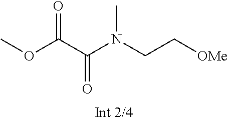

The title compound was prepared similar as described for Intermediate 2. MS (ESI): m/z 198.3 [M+Na]⁺.

### Intermediate 2/5: Methyl 2-oxo-2-(5-azaspiro[2.4]heptan-5-yl)acetate (Int 2/5)

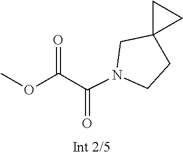

The title compound was prepared similar as described for Intermediate 2. MS (ESI): m/z 206.3 [M+Na]⁺.

### Intermediate 2/6: Methyl 2-(2-methylazetidin-1-yl)-2-oxoacetate (Int 2/6)

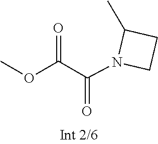

The title compound was prepared similar as described for Intermediate 2. MS (ESI): m/z 180.2 [M+Na]⁺.

### Intermediate 2/7: Methyl 2-(azetidin-1-yl)-2-oxoacetate (Int 2/7)

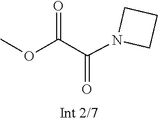

The title compound was prepared similar as described for Intermediate 2. MS (ESI): m/z 144.2 [M+H]⁺.

### Intermediate 3: 2-((2-(5-Cyano-2-(trifluoromethyl)phenyl)-1H-pyrrolo[2,3-b]pyridin-6-yl)amino)-2-oxoacetic acid (Int 3)

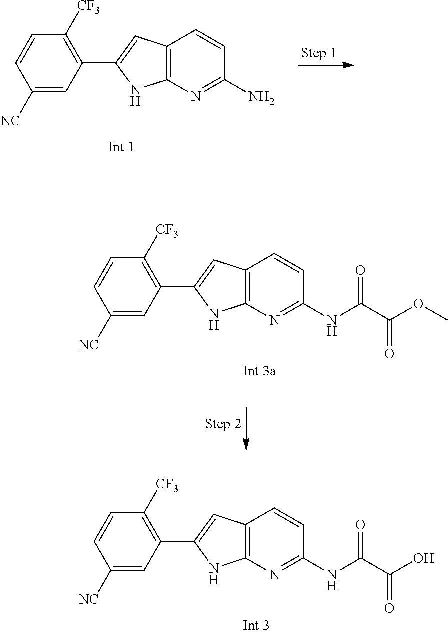 [SMILES: N#Cc1ccc(C(F)(F)F)c(-c2cc3ccc(N)nc3[nH]2)c1 ; COC(=O)C(=O)Nc1ccc2cc(-c3cc(C#N)ccc3C(F)(F)F)[nH]c2n1 ; N#Cc1ccc(C(F)(F)F)c(-c2cc3ccc(NC(=O)C(=O)O)nc3[nH]2)c1]

### Step 1: Methyl 2-((2-(5-Cyano-2-(trifluoromethyl)phenyl)-1H-pyrrolo[2,3-b]pyridin-6-yl)amino)-2-oxoacetate (Int 3a)

To a solution of 3-(6-amino-1H-pyrrolo[2,3-b]pyridin-2-yl)-4-(trifluoromethyl)benzonitrile (Int 1) (20 mg, 0.066 mmol) and triethylamine (14 μL, 0.099 mmol) in 0.3 mL DCM 2-chloro-2-oxoacetate (7 μL, 0.069 mmol) was added at 0° C. The mixture was stirred for 2.5 h at rt. The mixture was poured on sat. aq. NaHCO₃ (15 mL). The mixture was extracted with DCM (3×15 mL). The combined organic layers were washed with brine, dried over MgSO₄, filtered and concentrated under reduced pressure to afford the title compound as a slightly yellow solid. MS (ESI): m/z 389.4 [M+H]⁺.

### Step 2: 2-((2-(5-Cyano-2-(trifluoromethyl)phenyl)-1H-pyrrolo[2,3-b]pyridin-6-yl)amino)-2-oxoacetic acid (Int 3)

To a mixture of methyl 2-((2-(5-cyano-2-(trifluoromethyl)phenyl)-1H-pyrrolo[2,3-b]pyridin-6-yl)amino)-2-oxoacetate (Int 3a) in 0.6 mL THF/water (5:1) was added NaHCO₃ (3.8 mg, 0.045 mmol) and LiOH.H₂O (1.9 mg, 0.045 mmol). The mixture was stirred at rt for 1 h. The mixture was poured on 15 mL sat. NaHCO₃ and the mixture was extracted with DCM (3×15 mL). The combined organic layers were washed with brine, dried over MgSO₄, filtered and concentrated under reduced pressure to afford the title compound as a slightly yellow solid. MS (ESI): m/z 375.3 [M+H]⁺.

### EXAMPLE 1

### N-(2-(5-Cyano-2-(trifluoromethyl)phenyl)-1H-pyrrolo[2,3-b]pyridin-6-yl)-2-oxo-2-(pyrrolidin-1-yl)acetamide (1)

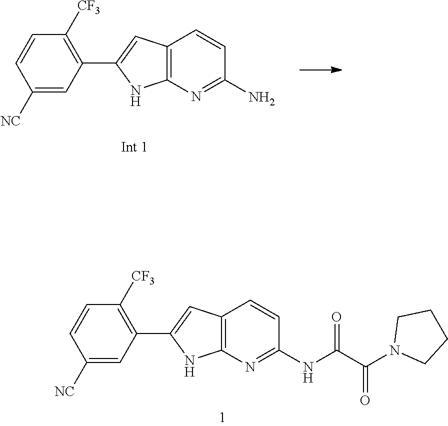 [SMILES: N#Cc1ccc(C(F)(F)F)c(-c2cc3ccc(N)nc3[nH]2)c1 ; N#Cc1ccc(C(F)(F)F)c(-c2cc3ccc(NC(=O)C(=O)N4CCCC4)nc3[nH]2)c1]

To a stirred mixture of 3-(6-amino-1H-pyrrolo[2,3-b]pyridin-2-yl)-4-(trifluoromethyl)benzonitrile (Int 1, 30 mg, 0.10 mmol) and methyl 2-oxo-2-(pyrrolidin-1-yl)acetate (It 2/1, 30 mg, 0.19 mmol, 1.9 eq.) in anhydrous DMA (150 μL) at 0° C. under nitrogen was added potassium methoxide (24 mg, 0.25 mmol, 3.5 eq.) upon which the mixture turned deep red. After additional stirring for 1.5 h at 0° C. the mixture was diluted with ACN and water (1:1, v/v, 2 mL) and the product was purified by prep. RP-HPLC, using water (0.1% formic acid) and ACN (0.1% formic acid) as eluents. The pooled product fractions were lyophilized to afford the title compound as a light yellow powder. ¹H-NMR (300 MHz, DMSO-ds): δ ppm 12.08 (s, 1H), 10.53 (s, 1H), 8.25 (s, 1H), 8.19-8.02 (m, 3H), 7.91-7.81 (d, J=8.3 Hz, 1H), 6.65 (s, 1H), 3.75-3.64 (i, 2H), 3.48-3.39 (m, 2H), 1.96-1.79 (m, 4H). MS (ESI): m/z 428.4 [M+H]⁺.

### EXAMPLES 1/1 TO 1/2

The following Examples were prepared similar as described for Example 1 using the appropriate intermediates.

| # | Int. # | Structure | Analytical data |
|---|---|---|---|
| 1/1 | Int 2/1 | 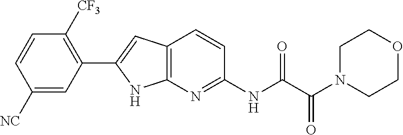 [SMILES: N#Cc1ccc(C(F)(F)F)c(-c2cc3ccc(NC(=O)C(=O)N4CCOCC4)nc3[nH]2)c1] | ¹H-NMR (300 MHz, DMSO-d₆, 320 K): δ ppm 11.92 (s br, 1H), 10.90 (s, 1H), 8.22 (s, 1H), 8.18-8.01 (m, 3H), 7.85 (s br, 1H), 6.64 (s, 1H), 3.73- 3.61 (m, 4H), 3.59- |
| | | | 3.49 (m, 4H). MS (ESI): |
| | | | m/z 444.4 [M + H]⁺. |
| 1/2 | Int 2/2 | 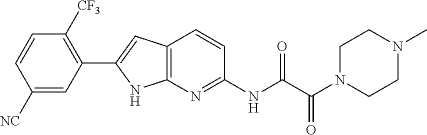 [SMILES: CN1CCN(C(=O)C(=O)Nc2ccc3cc(-c4cc(C#N)ccc4C(F)(F)F)[nH]c3n2)CC1] | ¹H-NMR (300 MHz, DMSO-d₆, 320 K): δ ppm 11.9 (s br, 1H), 10.87 (s, 1H), 8.20 (s, 1H), 8.17-7.99 (m, 3H), 7.82 (s br, 1H), 6.63 (s, 1H), 3.58- 3.50 (m, 2H), 3.49- |
| | | | 3.42 (m, 2H), 2.44- |
| | | | 2.29 (m, 4H), 2.21 (s, |
| | | | 3H). MS (ESI): m/z |
| | | | 457.4 [M + H]⁺. |
| 1/3 | Int 2/3 | 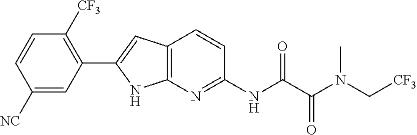 | ¹H-NMR (300 MHz, DMSO-d₆, 320 K): δ ppm 11.95 (s br, 1H), 11.04 (s br, 1H), 8.22 (s, 1H), 8.17-8.05 (m, 3H), 7.81 (s, 1H), 4.53 (s, 1H), 4.60-4.22 (m, 2H), 3.17 and 3.08 (s, 3H, conformers). MS |
| | | | (ESI): m/z 470.3 |
| | | | [M + H]⁺. |
| 1/4 | Int 2/4 | 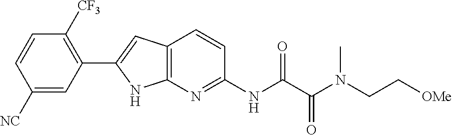 [SMILES: COCCN(C)C(=O)C(=O)Nc1ccc2cc(-c3cc(C#N)ccc3C(F)(F)F)[nH]c2n1] | ¹H-NMR (300 MHz, DMSO-d₆, 320 K): δ ppm 11.95 (s br, 1H), 10.84 and 10.69 (s br, 1H, conformers), 8.21 (s, 1H), 8.16-8.02 (m, 3H), 7.82 (s br, 1H), 6.65 (s, 1H), 3.64-3.52 (m, 4H), 3.34-2.96 (m, |
| | | | 6H). MS (ESI): m/z |
| | | | 446.4 [M + H]⁺. |
| 1/5 | Int 2/5 | 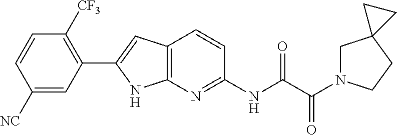 [SMILES: N#Cc1ccc(C(F)(F)F)c(-c2cc3ccc(NC(=O)C(=O)N4CCC5(CC5)C4)nc3[nH]2)c1] | ¹H-NMR (300 MHz, DMSO-d₆, 298 K): δ ppm 12.07 (s br, 1H), 10.55 and 10.48 (s br, 1H, conformers), 8.25 (s, 1H), 8.18-8.04 (m, 3H), 7.90-7.82 (m, 1H), 6.66 (s, 1H), 3.93-3.84 |
| | | | (m, 1H), 3.68-3.58 (m, |
| | | | 2H), 3.38-3.35 (m, 1H), |
| | | | 1.91-1.78 (m, 2H), |
| | | | 0.67-0.56 (m, 4H). MS |
| | | | (ESI): m/z 454.4 |
| | | | [M + H]⁺. |
| 1/6 | Int 2/6 | 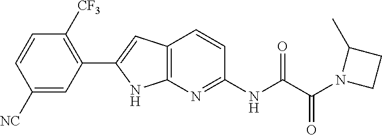 | ¹H-NMR (300 MHz, MeOD, 298 K): δ ppm 8.10-7.97 (m, 5H), 6.68 (s, 1H), 5.18-4.03 (m, 3H, conformers), 2.75- 1.92 (m, 2H, conformers), 1.77 and 1.56 (d, J = 6.3 Hz, 3H, conformers). MS (ESI): |
| | | | m/z 428.4 [M + H]⁺. |
| 1/7 | Int 2/7 | 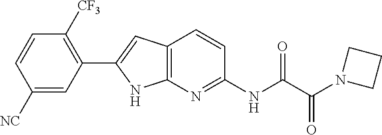 [SMILES: N#Cc1ccc(C(F)(F)F)c(-c2cc3ccc(NC(=O)C(=O)N4CCC4)nc3[nH]2)c1] | ¹H-NMR (300 MHz, MeOD, 298 K): δ ppm 8.10-7.98 (m, 5H), 6.69 (s, 1H), 4.81-4.73 (m, 2H), 4.26-4.19 (m, 2H), 2.51-2.39 (m, 2H). MS (ESI): m/z 414.4 [M + H]⁺. |

### EXAMPLE 2

### N-(2-(5-Cyano-2-(trifluoromethyl)phenyl)-1H-pyrrolo[2,3-b]pyridin-6-yl)-2-(3-hydroxy-3-methylazetidin-1-yl)-2-oxoacetamide (2)

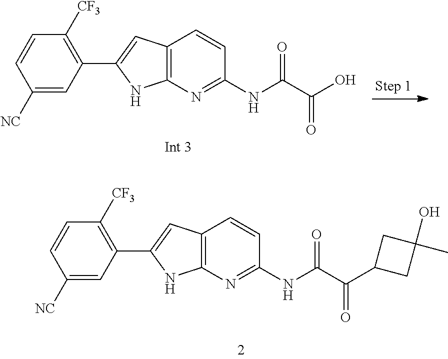 [SMILES: N#Cc1ccc(C(F)(F)F)c(-c2cc3ccc(NC(=O)C(=O)O)nc3[nH]2)c1 ; CC1(O)CC(C(=O)C(=O)Nc2ccc3cc(-c4cc(C#N)ccc4C(F)(F)F)[nH]c3n2)C1]

A solution of 2-((2-(5-cyano-2-(trifluoromethyl)phenyl)-1H-pyrrolo[2,3-b]pyridin-6-yl)amino)-2-oxoacetic acid (Int 3) (10.0 mg, 0.027 mmol), HOBt (3.8 mg, 0.028 mmol). EDC (5.4 mg, 0.028 mmol) and triethylamine (7 μL, 0.053 mmol) in 0.3 mL THF was stirred for 10 min at 0° C. 3-Methylazetidin-3-ol hydrochloride (3.5 mg, 0.028 mmol) was added and the mixture was stirred for 1 h at rt and for 1.5 h at 40° C. The mixture was concentrated under reduced pressure and the residue was purified by RP-HPLC using water (0.1% formic acid) and ACN (0.1% formic acid) to afford the title compound as a white solid. ¹H-NMR (300 MHz, DMSO-de, 298 K): δ ppm 12.10 (s br, 1H), 10.04 (s br, 1H), 8.25 (s, 1H), 8.20-8.07 (m, 3H), 7.84 (d, J=8.6 Hz, 1H), 6.67 (s, 1H), 5.79 (s, 1H), 4.48-4.32 (m, 2H), 3.99-3.85 (m, 2H), 1.42 (s, 3H). MS (ESI): m/z 444.4 [M+H]⁺.

### EXAMPLE 2/1

### N¹-Benzyl-N²-(2-(5-cyano-2-(trifluoromethyl)phenyl)-1H-pyrrolo[2,3-b]pyridin-6-yl)oxalamide (2/1)

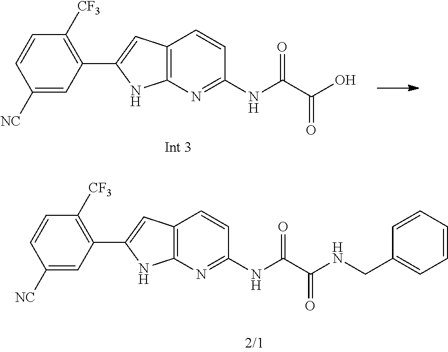 [SMILES: N#Cc1ccc(C(F)(F)F)c(-c2cc3ccc(NC(=O)C(=O)O)nc3[nH]2)c1 ; N#Cc1ccc(C(F)(F)F)c(-c2cc3ccc(NC(=O)C(=O)NCc4ccccc4)nc3[nH]2)c1]

The title compound was prepared similar as described for Example 2 using the appropriate intermediates. ¹H-NMR (300 MHz, MeOD, 298 K): δ ppm 8.11-7.98 (m, 5H), 7.41-7.25 (m, 5H), 6.69 (s, 1H), 4.55 (s, 2H). MS (ESI): m/z 464.4 [M+H]⁺.

The following compounds can be prepared by using similar procedures as those described above:

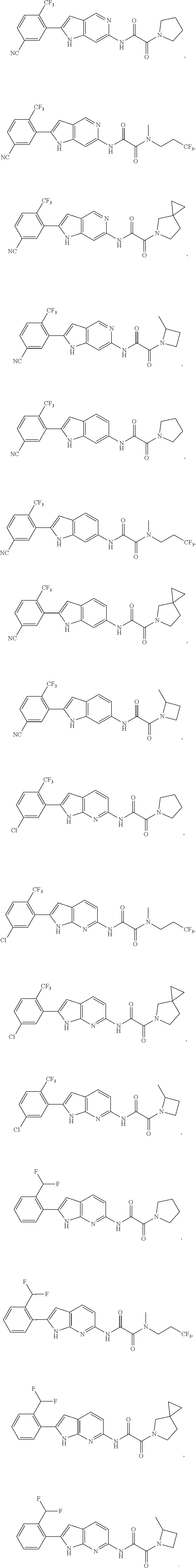

### Biological Assay

### AhR Direct Luciferase Reporter Assay in HepG2 Cells.

A stable cell line (HepG2 CYP1A1-LUC) was used in which part of the promoter region of the human CYP1A1 gene is stably integrated into the genome of human HepG2 hepatocytes (DSZM #ACC 180) in front of a Photinus pyralis Firefly Luciferase gene. A 1210 bp fragment comprising part of the human CYP1A1 promoter was isolated via SacI and BgIII restriction digestion from Lightswitch Clone S714555 (SwitchGearGenomics) and inserted between the SacI and BgIII sites in pGL4.30 (Promega #E8481) in front of the Firefly Luciferase gene. The resulting vector was linearized with NotI, transfected into HepG2 cells (DSMZ #ACC 180) and stably transfected clones selected with 250 μg/ml Hygromycin B. After repetitive rounds of subcloning and testing for robustly regulated luciferase activity after AhR agonist stimulation, a stable clonal HepG2 CYP1A1-Luc cell line was selected.

The HepG2 CYP1A1-Luc cells do express basal luciferase activity that can be increased via potent AhR agonists or decreased via potent AhR antagonists, added to the growth medium of the cells.

In typical reporter assays performed with this cell line, cells are grown in 96-well plates and AhR modulators are titrated into the growth medium in serial dilutions in RPMI-1640 Medium (Sigma #R7509) supplemented with 8.6% fetal calf serum (Sigma #F7524) and containing either no exogenous AhR agonist or 10 nM of the potent AhR agonist VAF347 (Calbiochem #182690). Cells are further cultivated for 18 hours and luciferase activities are determined from extracts of cells in buffers containing D-Luciferine and ATP using a LUMIstar Optima microplate Luminometer from BMG Labtech.

The AhR antagonistic potency of the example compounds is shown in Table 1 below (A=IC₅₀<100 nM, B=IC₅₀ 100 nM-1 μM, C=IC₅₀>1 μM)

TABLE 1

| | AhR |
|---|---|
| Example # | potency |
| 1 | A |
| 1/1 | B |
| 1/2 | C |
| 1/3 | A |
| 1/4 | B |
| 1/5 | A |
| 1/6 | A |
| 1/7 | B |
| 2 | B |
| 2/1 | B |

1. A compound represented by Formulae (I) to (III), an enantiomer, diastereomer, tautomer, solvate, N-oxide, prodrug or pharmaceutical acceptable salt thereof 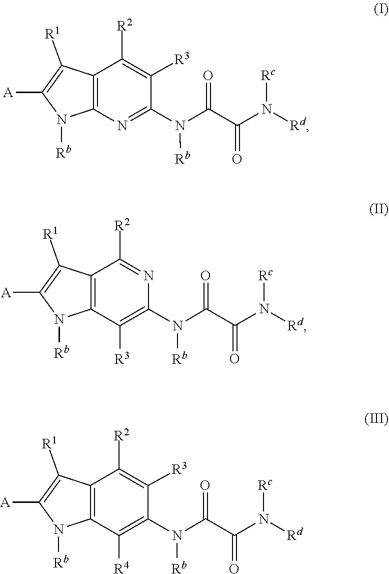
wherein
A represents a 6- to 10-membered mono- or bicyclic aryl or a 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, oxo, C(O)OR^{a}, OC(O)R^{a}, S(O)ₓ—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, oxo, OH, C₁₋₆-alkyl and halo-C₁₋₆-alkyl;
R^{a} is independently selected from hydrogen and C₁₋₆-alkyl,
wherein alkyl is unsubstituted or substituted with 1 to 3 substituents independently selected from the group consisting of halogen, halo-C₁₋₃-alkyl, OR^{b} and CN,
or
two R^{a} when taken together with the nitrogen to which they are attached complete a 4- to 8-membered ring containing carbon atoms and optionally containing 1 or 2 heteroatoms independently selected from the group consisting of O, S, and N,
wherein the 4- to 8-membered ring is unsubstituted or substituted with 1 to 3 substituents independently selected from the group consisting of halogen, halo-C₁₋₃-alkyl, OH, OR^{b} or CN;
R¹, R², R³ and R⁴ are independently selected from the group consisting of hydrogen, halogen, C₁₋₄-alkyl, halo-C₁₋₃-alkyl, OH, O—C₁₋₃-alkyl and CN;
R^{b} is independently selected from hydrogen and C₁₋₆-alkyl;
R^{c} represents hydrogen,
C₁₋₆-alkyl,
wherein the C₁₋₆-alkyl is unsubstituted or substituted with 1 to 3 substituents independently selected from halo, CN, oxo, OR^{b}, halo-C₁₋₆-alkyl, C(O)OR^{a}, OC(O)R^{a}, S(O)ₓ—C₁₋₆-alkyl, S(O)(═NR^{b})—C₁₋₆-alkyl, N(R^{a})₂, C(O)N(R^{a})₂, NR^{a}C(O)—C₁₋₆-alkyl, NR^{a}C(O)N(R^{a})₂, S(O)₂N(R^{a})₂, NR^{a}S(O)₂—C₁₋₆-alkyl, NR^{a}S(O)₂N(R^{a})₂, X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), or X—C₀₋₂-alkylene-(5- to 10-membered mono- or bicyclic heteroaryl containing 1 to 4 heteroatoms selected from N, O and S),
C₃₋₁₀-cycloalkyl,
3- to 10-membered heterocycloalkyl containing 1 to 4 heteroatoms independently selected from N, O and S,
6- to 10-membered mono- or bicyclic aryl,
5- to 10-membered mono- or bicyclic heteroaryl containing 1 to 4 heteroatoms independently selected from N, O and S,
C₁₋₆-alkylene-C₃₋₁₀-cycloalkyl,
C₁₋₆-alkylene-C₃₋₁₀-heterocycloalkyl,
C₁₋₆-alkylene-(6-10-membered mono- or bicyclic aryl), and
C₁₋₆-alkylene-(5- to 10-membered mono- or bicyclic heteroaryl containing 1 to 4 heteroatoms selected from N, O and S),
wherein alkylene is unsubstituted or substituted with 1 to 3 substituents independently selected from the group consisting of halogen, CN, OH, OR^{b}, oxo, C₁₋₆-alkyl and C₁₋₆-haloalkyl,
wherein cycloalkyl, heterocycloalkyl, aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a}, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo,
or
wherein two substituents on the cycloalkyl, heterocycloalkyl, aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl,
or
wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N;
R^{d} represents a C₁₋₆-alkyl,
wherein the C₁₋₆-alkyl is substituted with 1 to 3 substituents independently selected from halo, CN, oxo, OR^{b}, halo-C₁₋₆-alkyl, C(O)OR^{a}, OC(O)R^{a}, S(O)ₓ—C₁₋₆-alkyl, S(O)(═NR)—C₁₋₆-alkyl, N(R^{a})₂, C(O)N(R^{a})₂, NR^{a}C(O)—C₁₋₆-alkyl, NR^{a}C(O)N(R^{a})₂, S(O)₂N(R^{a})₂, NR^{a}S(O)₂—C₁₋₆-alkyl, NR^{a}S(O)₂N(R^{a})₂, X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), or X—C₀₋₂-alkylene-(5- to 10-membered mono- or bicyclic heteroaryl containing 1 to 4 heteroatoms selected from N, O and S),
C₃₋₁₀-cycloalkyl,
3- to 10-membered heterocycloalkyl containing 1 to 4 heteroatoms independently selected from N, O and S,
6- to 10-membered mono- or bicyclic aryl,
5- to 10-membered mono- or bicyclic heteroaryl containing 1 to 4 heteroatoms independently selected from N, O and S,
C₁₋₆-alkylene-C₃₋₁₀-cycloalkyl,
C₁₋₆-alkylene-C₃₋₁₀-heterocycloalkyl,
C₁₋₆-alkylene-(6-10-membered mono- or bicyclic aryl), and
C₁₋₆-alkylene-(5- to 10-membered mono- or bicyclic heteroaryl containing 1 to 4 heteroatoms selected from N, O and S),
wherein alkylene is unsubstituted or substituted with 1 to 3 substituents independently selected from the group consisting of halogen, CN, OH, OR^{b}, oxo, C₁₋₆-alkyl and C₁₋₆-haloalkyl,
wherein cycloalkyl, heterocycloalkyl, aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo,
or
wherein two substituents on the cycloalkyl, heterocycloalkyl, aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl,
or
wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N;
or
R^{c} and R^{d} when taken together with the nitrogen to which they are attached complete a 4- to 8-membered saturated or partially unsaturated ring containing carbon atoms and optionally containing 1 or 2 heteroatoms independently selected from the group consisting of O, S, and N,
wherein the ring is unsubstituted or substituted with 1 to 7 substituents independently selected from the group consisting of halogen, C₀₋₄-alkylene-OH, C₀₋₄-alkylene-CN, C₁₋₆-alkyl, C₀₋₄-alkylene-O—C₁₋₆-alkyl, oxo, C₀₋₄-alkylene-C(O)OR^{a}, C₀₋₄-alkylene-OC(O)R^{a}, C₀₋₄-alkylene-S(O)ₓ—C₁₋₆-alkyl, C₀₋₄-alkylene-S(O)(═NR^{b})—C₁₋₆-alkyl, C₀₋₄-alkylene-N(R^{a})₂, C₀₋₄-alkylene-C(O)N(R^{a})₂, C₀₋₄-alkylene-NR^{a}C(O)—C₁₋₆-alkyl, C₀₋₆-alkylene-NR^{a}C(O)N(R^{a})₂, C₀₋₄-alkylene-S(O)₂N(R^{a})₂, C₀₋₄-alkylene-NR^{a}S(O)₂—C₁₋₆-alkyl, C₀₋₄-alkylene-NR^{a}S(O)₂N(R^{a})₂, C₀₋₄-alkylene-C₃₋₁₀-cycloalkyl, C₀₋₄-alkylene-C₃₋₁₀-heterocycloalkyl, C₀₋₄-alkylene-(6-10-membered mono- or bicyclic aryl), C₀₋₄-alkylene-(5- to 10-membered mono- or bicyclic heteroaryl containing 1 to 4 heteroatoms selected from N, O and S), X—C₀₋₂-alkylene-C₃₋₁₀-cycloalkyl, X—C₀₋₂-alkylene-C₃₋₁₀-heterocycloalkyl, X—C₀₋₂-alkylene-(6-10-membered mono- or bicyclic aryl), X—C₀₋₂-alkylene-(5- to 10-membered mono- or bicyclic heteroaryl containing 1 to 4 heteroatoms selected from N, O and S), spirocyclic fused C₃₋₇-cycloalkyl and spirocyclic fused 3- to 7-membered heterocycloalkyl containing 1 to 3 heteroatoms independently selected from the group consisting of O, S and N,
wherein alkylene, alkyl, cycloalkyl, heterocycloalkyl, aryl and heteroaryl are unsubstituted or substituted with 1 to 3 substituents independently selected from the group consisting of halogen, C₁₋₃-alkyl, halo-C₁₋₃-alkyl, OH, CN and oxo,
or
wherein two substituents on the 4- to 8-membered ring together with the atoms they are attached to may form a 3- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, oxo, OH, C₁₋₆-alkyl and halo-C₁₋₃-alkyl;
or
wherein two substituents on two adjacent carbon atoms on the 4- to 8-membered ring form together with the carbon atoms they are attached to a 5- or 6-membered aromatic ring optionally containing 1 or 2 heteroatoms independently selected from O, N and S,
wherein the (hetero)aromatic ring is unsubstituted or substituted with 1 to 4 substituents independently selected from the group consisting of halogen, oxo, OH, C₁₋₆-alkyl and halo-C₁₋₃-alkyl;
X represents —O—, —N(R^{b})—, or —S(O)ₓ—; and
x is 0, 1 or 2;
with the proviso that if R^{c} is hydrogen or unsubstituted C₁₋₆-alkyl then R^{d} is not C₃₋₁₀-cycloalkyl, 3- to 10-membered heterocycloalkyl containing 1 to 4 heteroatoms independently selected from N, O and S, 6- to 10-membered mono- or bicyclic aryl or a 5- to 10-membered mono- or bicyclic heteroaryl containing 1 to 4 heteroatoms independently selected from N, O and S.

2. The compound according to claim 1, wherein the compound is represented by structural Formula (I) 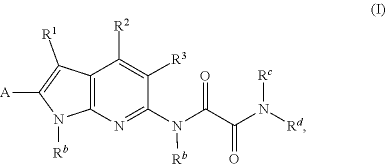
and an enantiomer, diastereomer, tautomer, solvate, N-oxide, prodrug or pharmaceutical acceptable salt thereof.

3. The compound according to claim 1, wherein
A is 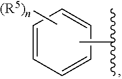
R⁵ is independently selected from halogen, OH, CN, C₁₋₆-alkyl, O—C₁₋₆-alkyl, and C₃₋₆-cycloalkyl,
wherein alkyl and cycloalkyl are unsubstituted or substituted with 1 to 3 substituents independently selected from the group consisting of halogen, C₁₋₃-alkyl and halo-C₁₋₃-alkyl; and
n is 0 to 3.

4. The compound according to claim 1, wherein
A is 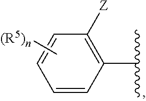
wherein
Z is F, Cl, CH₃, CH₂CH₃, CHF₂ or CF₃;
R⁵ is independently selected from halogen and CN; and
n is 0 to 2.

5. The compound according to a claim 1, wherein R^{h} is hydrogen.

6. The compound according to claim 1, wherein R¹, R², R³ and R⁴ are hydrogen.

7. The compound according to claim 1, wherein R^{c} represents hydrogen or C₁₋₆-alkyl, wherein the C₁₋₆-alkyl is unsubstituted or substituted with 1 to 3 substituents independently selected from halo, CN, oxo, OR^{b} and halo-C₁₋₆-alkyl.

8. The compound according to claim 1, wherein R^{d} represents
C₁₋₆-alkyl, wherein the C₁₋₆-alkyl is substituted with 1 to 3 substituents independently selected from halo, CN, oxo, OR^{b}, halo-C₁₋₆-alkyl, X—C₀₋₂-alkylene-C₃₋₆-cycloalkyl, X—C₀₋₂-alkylene-C₃₋₈-heterocycloalkyl, X—C₀₋₂-alkylene-phenyl, or X—C₀₋₂-alkylene-(5- to 8-membered mono- or bicyclic heteroaryl containing 1 to 4 heteroatoms selected from N, O and S),
C₃₋₆-cycloalkyl,
3- to 8-membered heterocycloalkyl containing 1 to 4 heteroatoms independently selected from N, O and S,
phenyl,
5- to 8-membered mono- or bicyclic heteroaryl containing 1 to 4 heteroatoms independently selected from N, O and S,
C₁₋₄-alkylene-C₃₋₆-cycloalkyl,
C₁₋₄-alkylene-C₃₋₁₀-heterocycloalkyl,
C₁₋₄-alkylene-phenyl, and
C₁₋₄-alkylene-(5- to 8-membered mono- or bicyclic heteroaryl containing 1 to 4 heteroatoms selected from N, O and S),
wherein alkylene is unsubstituted or substituted with 1 to 3 C₁₋₃-alkyl groups,
wherein cycloalkyl, heterocycloalkyl, phenyl and heteroaryl are unsubstituted or substituted with 1 to 7 substituents independently selected from the group consisting of halogen, OH, CN and O—C₁₋₆-alkyl,
or wherein two substituents on the cycloalkyl, heterocycloalkyl, phenyl or heteroaryl group together with the atoms they are attached to may form a 5- to 7-membered saturated or partially unsaturated carbocyclic ring or heterocyclic ring containing 1 to 3 heteroatoms independently selected from O, N and S,
or wherein two substituents on the cycloalkyl or heterocycloalkyl group together with the atom they are attached to may form a spirocyclic fused C₃₋₇-cycloalkyl or spirocyclic fused 3- to 7-membered heterocycloalkyl containing 1 to 3 heteroatoms independently selected from the group consisting of O, S and N.

9. The compound according to claim 1, wherein 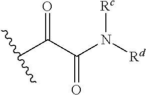
is 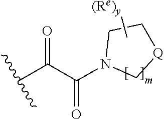
wherein
R^{e} is independently selected from the group consisting of halogen, CN, OH, O—C₁₋₆-alkyl, C₁₋₆-alkyl, spirocyclic fused C₃₋₇-cycloalkyl and spirocyclic fused 3- to 7-membered heterocycloalkyl containing 1 heteroatom selected from the group consisting of O, S and N;
Q is CH₂, CHR^{e}, O, NH, N—C₁₋₆-alkyl or S;
m is 0, 1 or 2; and
y is 0, 1 or 2.

10. The compound according to claim 1, which is selected from the group consisting of 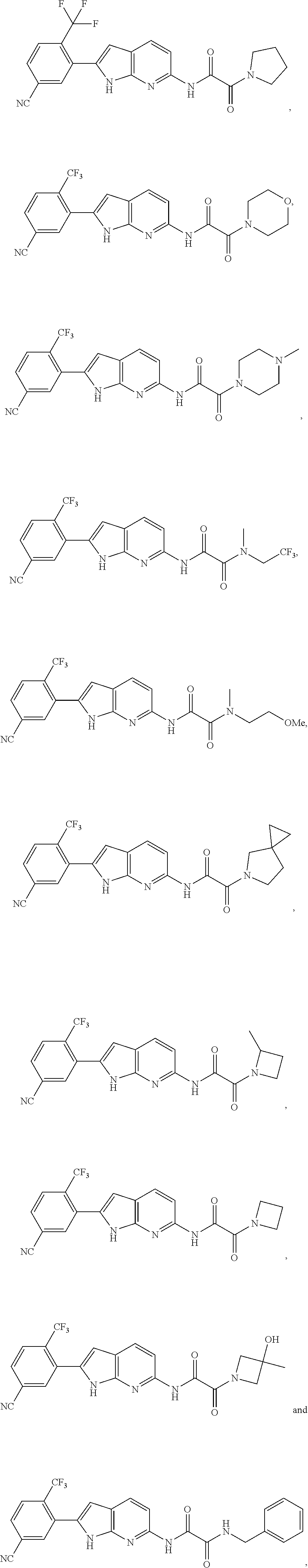
and an enantiomer, diastereomer, tautomer, solvate, N-oxide, prodrug or pharmaceutical acceptable salt thereof.

11. A pharmaceutical composition comprising the compound according to claim 1 and a physiologically acceptable excipient.

12. (canceled)

13. A method for the prophylaxis and/or treatment of a disease or condition mediated by aryl hydrocarbon receptor (AhR), comprising administering to a subject in need thereof a therapeutically effective amount of a compound of claim 1, or an enantiomer, diastereomer, tautomer, solvate, N-oxide, prodrug or pharmaceutical acceptable salt thereof.

14. The method according to claim 13, wherein the disease or condition mediated by aryl hydrocarbon receptor (AhR) is cancer.

15. The method according to claim 14, wherein the compound is administered with one or more therapeutic agents for cancer selected from the group consisting of PD-1 agent, PD-L1 agent, CTLA-4 agent, IDO1 inhibitor, chemotherapeutic agent, anticancer vaccine, Toll like receptor agonist, oncolytic virus, STING agonist and cytokine therapy, or wherein the compound is administered under irradiation therapy.

16. A method for the prophylaxis and/or treatment of a disease or condition mediated by aryl hydrocarbon receptor (AhR), comprising administering to a subject in need thereof a therapeutically effective amount of a pharmaceutical composition of claim 11.

17. The method according to claim 16, wherein the disease or condition mediated by aryl hydrocarbon receptor (AhR) is cancer.

18. The method according to claim 16, wherein the compound is administered with one or more therapeutic agents for cancer selected from the group consisting of PD-1 agent, PD-L1 agent, CTLA-4 agent, IDO1 inhibitor, chemotherapeutic agent, anticancer vaccine, Toll like receptor agonist, oncolytic virus, STING agonist and cytokine therapy, or wherein the compound is administered under irradiation therapy.
